(19) United States
(12) Patent Application Publication
(10) Pub. No.: US 2020/0411192 A1
(43) Pub. Date: Dec. 31, 2020

(54) DISEASE VULNERABILITY PREDICTION USING CONTEXTUAL DEVICES

(71) Applicant: INTERNATIONAL BUSINESS MACHINES CORPORATION, Armonk, NY (US)

(72) Inventors: Craig M. TRIM, Ventura, CA (US); Stefan VAN DER STOCKT, Austin, TX (US); Jeremy R. FOX, Georgetown, TX (US); Sarbajit K. RAKSHIT, Kolkata (IN)

(21) Appl. No.: 16/452,871

(22) Filed: Jun. 26, 2019

(51) Int. Cl. G16H 50/30; G16H 20/70; G16H 10/60

(52) U.S. Cl. CPC G16H 50/30; G16H 10/60; G16H 20/70

(57) ABSTRACT

Methods and systems for visually disease vulnerability prediction using contextual devices are disclosed. A computer-implemented method includes: determining, by a computing device, information about a patient including a current disease and contextual parameters; determining, by the computing device, a vulnerability score indicating a predicted vulnerability of the patient to at least one associated disease, based on the information about the patient; determining, by the computing device, a surrounding context for the patient, based on the information about the patient; and determining, by the computing device, a treatment for the patient, based on the information about the patient.

### BACKGROUND

The present invention generally relates to computing devices and, more particularly, to methods and systems for disease vulnerability prediction using contextual devices.

A patient diagnosis may initially reflect only one disease. However, during the course of treatment for a diagnosed disease, a patient may contract another disease. When a patient has multiple diseases, the treatment of one disease may require prioritization over the treatment of another disease.

### SUMMARY

In a first aspect of the invention, there is a computer-implemented method that includes: determining, by a computing device, information about a patient including a current disease and contextual parameters; determining, by the computing device, a vulnerability score indicating a predicted vulnerability of the patient to at least one associated disease, based on the information about the patient; determining, by the computing device, a surrounding context for the patient, based on the information about the patient; and determining, by the computing device, a treatment for the patient, based on the information about the patient.

In another aspect of the invention, there is a computer program product that includes a computer readable storage medium having program instructions embodied therewith. The program instructions are executable by a computing device to cause the computing device to: determine diseases from a plurality of diseases that commonly occur together using clustering; determine individual risk factors of each of the plurality of diseases using a data pipeline; determine common risk factors of the plurality of diseases; and determine a probability of two diseases occurring together in a patient using the determined diseases that commonly occur together, the determined individual risk factors, and the determined common risk factors.

In another aspect of the invention, there is a system that includes: a hardware processor, a computer readable memory, and a computer readable storage medium associated with a computing device; program instructions to determine information about a patient including a current disease and contextual parameters; program instructions to determine a vulnerability score indicating a predicted vulnerability of the patient to at least one associated disease, based on the information about the patient; program instructions to determine a surrounding context for the patient, based on the information about the patient; and program instructions to determine a treatment for the patient, based on the information about the patient, wherein the program instructions are stored on the computer readable storage medium for execution by the hardware processor via the computer readable memory.

### BRIEF DESCRIPTION OF THE DRAWINGS

The present invention is described in the detailed description which follows, in reference to the noted plurality of drawings by way of non-limiting examples of exemplary embodiments of the present invention.

FIG. 1 depicts a computer system in accordance with aspects of the invention.

FIG. 2 depicts an illustrative environment in accordance with aspects of the invention.

FIGS. 3, 4, and 5 depict flowcharts of exemplary methods performed in accordance with aspects of the invention.

### DETAILED DESCRIPTION

The present invention generally relates to computing devices and, more particularly, to methods and systems for disease vulnerability prediction using contextual devices. As described herein, aspects of the invention include a method and system for determining diseases that commonly occur together using clustering, determining individual risk factors of each of the diseases using a pipeline of data mining approaches, determining common risk factors using an automated procedure, and predicting the possibility of two diseases occurring together in a patient using a multivariate model.

Conventional trained models for predicting specific diseases typically do not account for patients that suffer from not only one disease but from one or more other diseases as well. These other diseases may be related (or reciprocal) or completely unrelated. A patient diagnosis may reflect only one disease. During the course of treatment, the patient may contract another disease. It becomes challenging for the physician involved to make correct decisions in this situation. In some cases, the treatment of one disease may need to be prioritized over the treatment of another disease.

Embodiments address these problems with conventional models for predicting diseases by providing methods and systems for identifying the contextual parameters around a given diagnosis and disease for a patient and using a model to predict the patient's vulnerability to other potential diseases. Accordingly, embodiments improve the functioning of a computer by providing an effective and comprehensive forecasting mechanism using a two-phase analysis procedure. In particular, embodiments improve software by providing methods and systems for determining diseases that commonly occur together using clustering. Additionally, embodiments improve software by providing methods and systems for determining individual risk factors of each of the diseases using a pipeline of data mining approaches. Additionally, embodiments improve software by providing methods and systems for determining common risk factors using an automated procedure. Additionally, embodiments improve software by providing methods and systems for predicting the possibility of two diseases occurring together in a patient using a multivariate model.

Accordingly, through the use of rules that improve computer-related technology, implementations of the invention allow computer performance of functions not previously performable by a computer. Additionally, implementations of the invention use techniques that are, by definition, rooted in computer technology (e.g., machine learning and artificial intelligence techniques, augmented reality, and data mining pipelines).

Aspects of the present invention identify the contextual parameters around a given diagnosis and disease for a patient. In particular, the contextual parameters that are identified include (1) physiological indicators, (2) psychological attributes and insights, and (3) past history. Additionally, aspects of the present invention use a model to predict a patient's vulnerability to other potential diseases. In particular, aspects of the present invention provide an effective and comprehensive forecasting mechanism that (1) uses clustering to determine diseases that commonly occur together, (2) uses a data mining pipeline to select individual risk factors of each of the diseases, (3) identifies common risk factors, and (4) predicts the possibility of two or more diseases occurring together in a patient.

In embodiments, using historical learning of associated diseases and side effects when one patient is being treated with another disease, an intelligent system (e.g., a system that uses artificial intelligence and machine learning techniques) predicts how any patient is vulnerable to another disease in different stages of the disease that is being treated. Additionally, in embodiments, the intelligent system considers the surrounding context (e.g., geographic location, exercise, diet, etc.) in the machine learning model to predict how the surrounding context is related to the associated diseases when the patient is being treated with another disease. Accordingly, the intelligent system recommends an appropriate surrounding context so that the associated diseases can be prevented or proactive measures can be taken.

Additionally, in embodiments, a machine learning model considers the patient's response, treatments (e.g., a medicine and dosage) applied while treating associated diseases, and how the associated disease makes the patient's condition more serious, and accordingly the intelligent system recommends an appropriate treatment to the patient when the patient is being treated with one disease. Additionally, in embodiments, when any patient is being treated with one disease, an augmented reality system is provided for use by the patient, doctor, medical staff, or family members that highlights a surrounding context to be avoided and/or a surrounding context to be adopted, in order to avoid associated diseases with the patient.

Accordingly, aspects of the present invention provide for identification of multiple human diseases through a correlation clustering data pipeline. Aspects of the present invention also provide for a data driven analytics based approach and deriving a predictive model for vulnerability assessment.

To the extent the implementations collect, store, or employ personal information (e.g., medical information) of individuals, it should be understood that such information shall be used in accordance with all applicable laws concerning protection of personal information. Additionally, the collection, storage, and use of such information, as well as, e.g., use of the disease vulnerability prediction methods and systems described herein, may be subject to advance notification and consent of the individual to such activity, for example, through “opt-in” or “opt-out” processes as may be appropriate for the situation and type of information. Storage and use of personal information may be in an appropriately secure manner reflective of the type of information, for example, through various encryption and anonymization techniques for particularly sensitive information.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

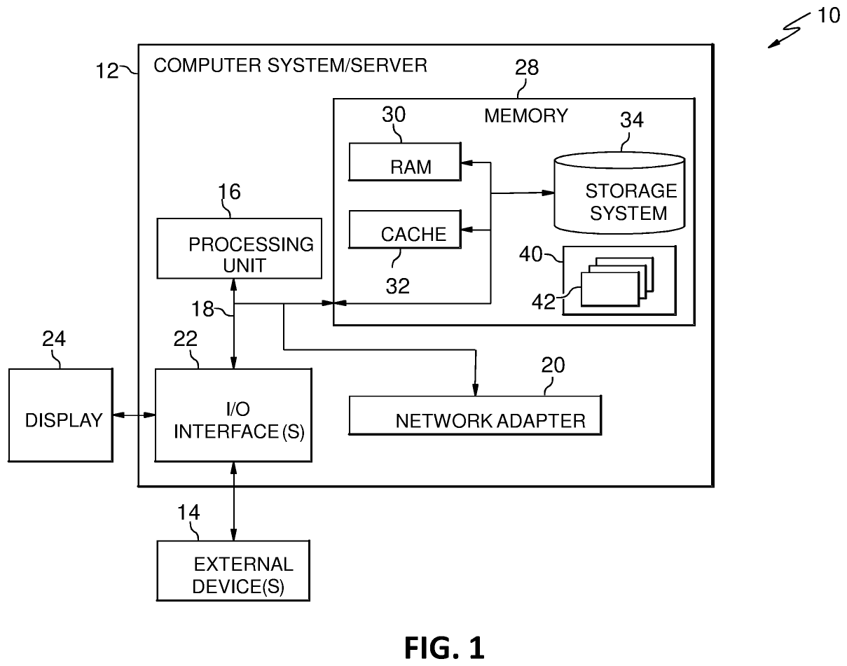

Referring now to FIG. 1, a schematic of an example of a computing infrastructure is shown. Computing infrastructure 10 is only one example of a suitable computing infrastructure and is not intended to suggest any limitation as to the scope of use or functionality of embodiments of the invention described herein. Regardless, computing infrastructure 10 is capable of being implemented and/or performing any of the functionality set forth hereinabove.

In computing infrastructure 10 there is a computer system (or server) 12, which is operational with numerous other general purpose or special purpose computing system environments or configurations. Examples of well-known computing systems, environments, and/or configurations that may be suitable for use with computer system 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 12 may be described in the general context of computer system executable instructions, such as program modules, being executed by a computer system. Generally, program modules may include routines, programs, objects, components, logic, data structures, and so on that perform particular tasks or implement particular abstract data types. Computer system 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system 12 in computing infrastructure 10 is shown in the form of a general-purpose computing device. The components of computer system 12 may include, but are not limited to, one or more processors or processing units (e.g., CPU) 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system 12 typically includes a variety of computer system readable media. Such media may be any available media that is accessible by computer system 12, and it includes both volatile and non-volatile media, removable and non-removable media.

System memory 28 can include computer system readable media in the form of volatile memory, such as random access memory (RAM) 30 and/or cache memory 32. Computer system 12 may further include other removable/non-removable, volatile/non-volatile computer system storage media. By way of example only, storage system 34 can be provided for reading from and writing to a nonremovable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system 12 may also communicate with one or more external devices 14 such as a keyboard, a pointing device, a display 24, etc.; one or more devices that enable a user to interact with computer system 12; and/or any devices (e.g., network card, modem, etc.) that enable computer system 12 to communicate with one or more other computing devices. Such communication can occur via Input/Output (I/O) interfaces 22. Still yet, computer system 12 can communicate with one or more networks such as a local area network (LAN), a general wide area network (WAN), and/or a public network (e.g., the Internet) via network adapter 20. As depicted, network adapter 20 communicates with the other components of computer system 12 via bus 18. It should be understood that although not shown, other hardware and/or software components could be used in conjunction with computer system 12. Examples, include, but are not limited to: microcode, device drivers, redundant processing units, external disk drive arrays, RAID systems, tape drives, and data archival storage systems, etc.

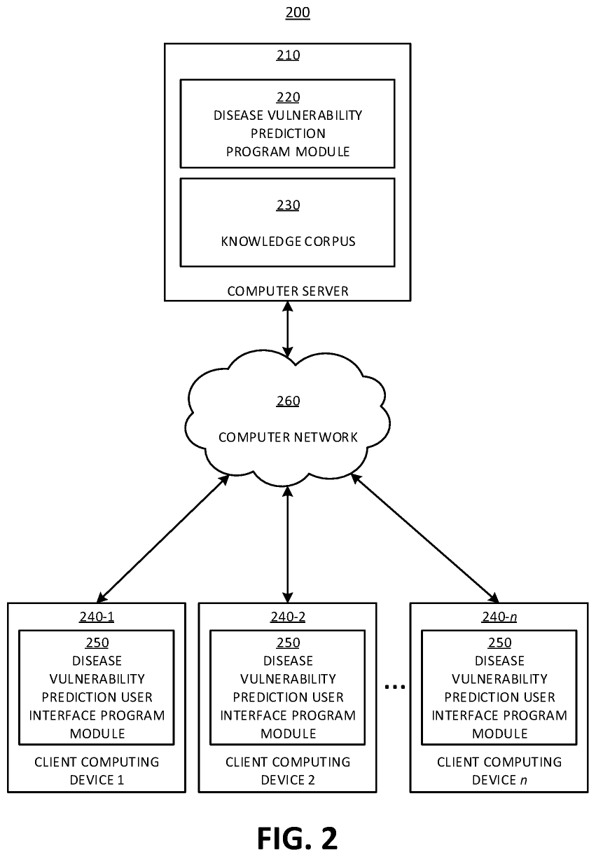

FIG. 2 depicts an illustrative environment 200 in accordance with aspects of the invention. As shown, the environment 200 comprises a computer server 210 and a plurality of client computing devices 240-1, 240-2, . . . , 240-n which are in communication via a computer network 260. In embodiments, the computer network 260 is any suitable network including any combination of a LAN, WAN, or the Internet. In embodiments, the computer server 210 and the plurality of client computing devices 240-1, 240-2, . . . , 240-n are physically collocated, or, more typically, are situated in separate physical locations.

The quantity of devices and/or networks in the environment 200 is not limited to what is shown in FIG. 2. In practice, the environment 200 may include additional devices and/or networks; fewer devices and/or networks; different devices and/or networks; or differently arranged devices and/or networks than illustrated in FIG. 2. Also, in some implementations, one or more of the devices of the environment 200 may perform one or more functions described as being performed by another one or more of the devices of the environment 200.

In embodiments, the computer server 210 is a computer device comprising one or more elements of the computer system/server 12 (as shown in FIG. 1). In particular, the computer server 210 is implemented as hardware and/or software using components such as mainframes; RISC (Reduced Instruction Set Computer) architecture based servers; servers; blade servers; storage devices; networks and networking components; virtual servers; virtual storage; virtual networks, including virtual private networks; virtual applications and operating systems; and virtual clients.

In embodiments, the computer server 210 includes a disease vulnerability prediction program module 220, which includes hardware and/or software such as one or more of the program modules 42 shown in FIG. 1. The disease vulnerability prediction program module 220 includes program instructions for determining diseases that commonly occur together using clustering, determining individual risk factors of each of the diseases using a pipeline of data mining approaches, determining common risk factors using an automated procedure, and predicting the possibility of two diseases occurring together in a patient using a multivariate model. In embodiments, the program instructions included in the disease vulnerability prediction program module 220 of the computer server 210 are executed by one or more hardware processors. In embodiments, the computer server 210 also includes knowledge corpus 230, which stores information used by the disease vulnerability prediction program module 220 for disease vulnerability prediction, as described below.

Still referring to FIG. 2, in embodiments, each of the plurality of user computing devices 240-1, 240-2, . . . , 240-n is a computer device comprising one or more elements of the computer system/server 12 (as shown in FIG. 1). In particular, the user computing device 420 is a desktop computer, a laptop computer, a mobile device such as a cellular phone, tablet, personal digital assistant (PDA), or other computing device.

In embodiments, each of the plurality of user computing devices 240-1, 240-2, . . . , 240-n includes disease vulnerability prediction user interface program module 250, which includes hardware and/or software such as one or more of the program modules 42 shown in FIG. 1. The disease vulnerability prediction user interface program module 250 includes program instructions for a user interface for the disease vulnerability prediction program module 220 of the computer server 210. In embodiments, the program instructions included in the disease vulnerability prediction user interface program module 250 of each of the plurality of user computing devices 240-1, 240-2, . . . , 240-n are executed by one or more hardware processors.

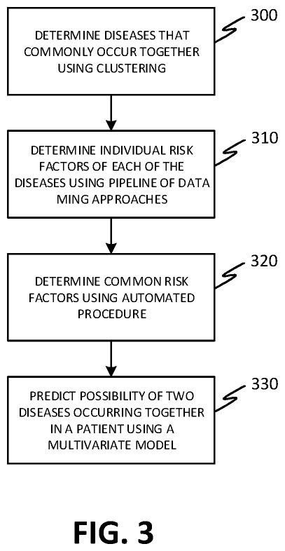

FIG. 3 depicts a flowchart of an exemplary method performed by the disease vulnerability prediction program module 220 of the computer server 210 in accordance with aspects of the invention. The steps of the method are performed in the environment of FIG. 2 and are described with reference to the elements shown in FIG. 2.

At step 300, the computer server 210 determines diseases that commonly occur together using clustering. In embodiments, step 300 comprises the disease vulnerability prediction program module 220 of the computer server 210 using a clustering method to determine diseases that commonly occur together.

In particular, referring to step 300, an optimal model for a data set involves a form of cluster analysis. This particular cluster analysis requires grouping a set of objects in such a way that objects in the same group (called a cluster) are more similar (in some sense) to each other than to those in other groups (clusters). The model created for this solution attempts to find clusters between several quantitative explanatory variables (e.g., contextual parameters including physiological indicators, psychological attributes and insights, and past history) and response variables (e.g., diseases). Clusters of a certain complexity and origin are located by the disease vulnerability prediction program module 220. These clusters are useful as a secondary mechanism for predicting the presence of multiple diseases in a patient.

Still referring to step 300, the disease vulnerability prediction program module 220 uses the following ranking method: (1) a clustering algorithm is run continuously with the omission of specific variables at each interval; and (2) initially, the variable exclusion is a function of both intuition and brute force combinatorics.

Still referring to step 300, the disease vulnerability prediction program module 220 tracks the following output: (1) Pseudo F Statistic, (2) Approximate Expected Over-All R-Squared, and (3) Cubic Clustering Criterion. In particular, the Pseudo F Statistic is a value that describes the ratio of between-cluster variance to within-cluster variance. The disease vulnerability prediction program module 220 finds a variable combination that maximizes this value once the minimum number of variables is selected. The Approximate Expected Over-All R-Squared is the “percent of variance explained” by the model. The disease vulnerability prediction program module 220 maximizes this value by the omission of as many variables as possible. Finally, the Cubic Clustering Criterion is tracked as a point of reference by the disease vulnerability prediction program module 220.

Still referring to step 300, the disease vulnerability prediction program module 220 uses a two-pass approach to find the clustering approach with the highest score. The first pass involves finding the variable combination with the highest Pseudo F Statistic. The disease vulnerability prediction program module 220 locates a variable combination with the most appropriate number of clusters. This second pass involves the disease vulnerability prediction program module 220 iterating through the number of possible clusters while running the algorithm and tracking the Approximate Expected Over-All R-Squared value. The highest value represents the best possible cluster number.

At step 310, the computer server 210 determines individual risk factors of each of the diseases using a pipeline of data mining approaches. (An individual risk factor is a contextual parameter that has a correlation with only one disease or condition.) In embodiments, step 310 comprises the disease vulnerability prediction program module 220 of the computer server 210 determining individual risk factors of each of the diseases using a pipeline of data mining approaches and the clusters generated at step 300.

In particular, at step 310, the disease vulnerability prediction program module 220 uses logistic regression for categorical target variables. The logistic model formula computes the probability of the selected disease x (where x ranges from a value of 0 if the patient does not suffer from the disease to a value of 1 if the patient suffers from the disease) as a function of the values of the predictive risk factors (contextual parameters). If the patient suffers from this disease, the conditional probability is given by p(y=1|X)=p(X), and the logistic model formula takes the following form:

$$y = log [ \frac{p ( X )}{1 - p ( X )} ] = {β}_{0} + {β}_{1} {x}_{1} + {β}_{2} {x}_{2} + , … , + {β}_{k} {x}_{k}$$

where X=(x₁, x₂, . . . , xₙ) represents the vector of k's risk factors selected by the logistic regression approach. Finally, the disease vulnerability prediction program module 220 obtains a logistic regression model with the best correct predictive rate, and the indicators in this model are the risk factors of the disease.

At step 320, the computer server 210 determines common risk factors using an automated procedure. (A common risk factor is a contextual parameter that has a correlation with more than one disease or condition.) In embodiments, step 310 comprises the disease vulnerability prediction program module 220 of the computer server 210 determining common risk factors using an automated procedure and the clusters generated at step 300.

In particular, at step 320, the disease vulnerability prediction program module 220 loads risk factors (contextual parameters) into a confusion matrix. (For example, old age, body mass index (BMI), and low educational attainment are statistically significant risk factors for hypertension). The disease vulnerability prediction program module 220 uses the confusion matrix in a logistic regression classifier to assist in the calculation of a cross-tabulation of observed (true) and predicted classes (model). Metrics including precision and recall are used by the disease vulnerability prediction program module 220 to aid in interpreting the accuracy of the model and choosing the most relevant risk factors (contextual parameters) common to the diseases in a given cluster.

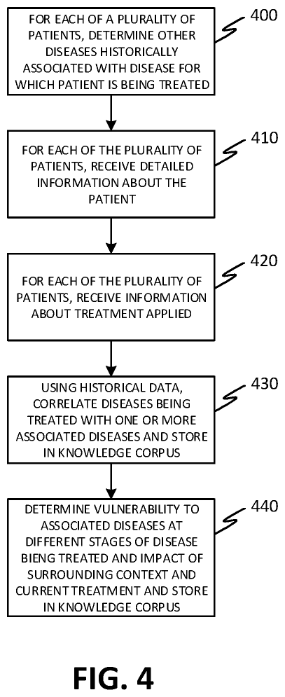

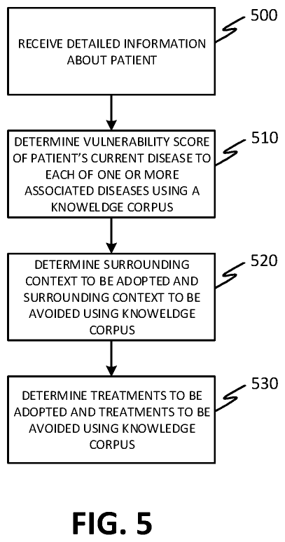

At step 330, the computer server 210 predicts the possibility of two diseases occurring together in a patient using a multivariate model. In embodiments, step 310 comprises the disease vulnerability prediction program module 220 of the computer server 210 predicting the possibility of two diseases occurring together in a patient using a multivariate model, in response to a request received from the disease vulnerability prediction user interface program module 250 of one of the client computing devices 240-1, 240-2, . . . , 240-n. In particular, at step 330, the disease vulnerability prediction program module 220 uses the clusters determined at step 300, the individual risk factors of each of the diseases determined at step 310, and the common risk factors determined at step 320 to create a multivariate model for predicting the possibility of these two diseases occurring together in a given patient (as described below with respect to FIG. 4) and uses the multivariate model to predict the possibility of two diseases occurring together in a patient (as described below with respect to FIG. 5). The prediction generated by the disease vulnerability prediction program module 220 of the computer server 210 is then sent to one of the client computing devices 240-1, 240-2, . . . , 240-n for display by the disease vulnerability prediction user interface program module 250 on a display of one of the client computing devices 240-1, 240-2, 240-n or an augmented reality system provided by one of the client computing devices 240-1, 240-2, 240-n.

Still referring to step 330, in embodiments, the disease vulnerability prediction program module 220 also determines surrounding context (e.g., geographic location, exercise, diet, etc.) to be adopted and/or surrounding context to be avoided. Additionally, in embodiments, the disease vulnerability prediction program module 220 determines a treatment (e.g., a medicine and dosage) to be adopted and/or treatments to be avoided. This information about surrounding context and/or treatments determined by the disease vulnerability prediction program module 220 of the computer server 210 is then sent to one of the client computing devices 240-1, 240-2, . . . , 240-n for display by the disease vulnerability prediction user interface program module 250. In embodiments, the disease vulnerability prediction user interface program module 250 displays the information about surrounding context and/or treatments on a display of one of the client computing devices 240-1, 240-2, 240-n or an augmented reality system provided by one of the client computing devices 240-1, 240-2, 240-n.

FIG. 4 depicts a flowchart of an exemplary method for creating a multivariate model performed by the disease vulnerability prediction program module 220 of the computer server 210 in accordance with aspects of the invention. The steps of the method are performed in the environment of FIG. 2 and are described with reference to the elements shown in FIG. 2.

At step 400, for each of a plurality of patients, the computer server 210 determines other diseases historically associated with a disease for which the patient is being treated. In embodiments, step 400 comprises the disease vulnerability prediction program module 220 of the computer server 210 determining, for each of a plurality of patients, other diseases historically associated with a disease for which the patient is being treated, using the clusters generated at step 300 (of FIG. 3).

At step 410, for each of the plurality of patients, the computer server 210 receives detailed information about the patient. In embodiments, step 410 comprises the disease vulnerability prediction program module 220 of the computer server 210 receiving, for each of a plurality of patients, detailed information about the patient, such as age, demographic information, current condition, current surroundings, geographic location, interaction with other people, etc., while the patient is being treated. In embodiments, the disease vulnerability prediction program module 220 aggregates and anonymizes the detailed information. The detailed information may be received by the disease vulnerability prediction program module 220 from cameras and Internet of Things (IoT) sensors (e.g., via the client computing devices 240-1, 240-2, . . . , 240-n) and analyzed using machine learning techniques to extract contextual parameters including physiological indicators, psychological attributes and insights, and past history.

At step 420, for each of the plurality of patients, the computer server 210 receives information about a treatment applied. In embodiments, step 420 comprises the disease vulnerability prediction program module 220 of the computer server 210 receiving, for each of a plurality of patients, information about a treatment applied (e.g., a medicine and a dosage) as well as information about treatment of associated diseases (e.g., via the client computing devices 240-1, 240-2, . . . , 240-n) and analyzing the information using machine learning techniques to extract contextual parameters.

At step 430, the computer server 210, using historical data, correlates diseases being treated with one or more associated diseases and stores the correlations in the knowledge corpus 230. In embodiments, step 430 comprises the disease vulnerability prediction program module 220 of the computer server 210 correlating one or more associated diseases with a disease that is being treated (based on the information received at step 400), contextual parameters (determined at step 410), and treatment applied (based on the information received at step 420), using the clusters from step 300 (of FIG. 3), the individual risk factors determined at step 310 (of FIG. 3), and the common risk factors determined at step 320 (of FIG. 3). The disease vulnerability prediction program module 220 then stores the correlated information in the knowledge corpus 230 of the computer server 210. This correlated information is usable by the disease vulnerability prediction program module 220 to predict associated diseases to which a particular patient being treated is vulnerable.

At step 440, the computer server 210 determines vulnerability to associated diseases at different stages of a disease being treated and an impact of surrounding context (e.g., geographic location, exercise, diet, etc.) and current treatment (e.g., a medicine and dosage), and stores information about the determined vulnerabilities and impacts in the knowledge corpus 230. In embodiments, step 430 comprises the disease vulnerability prediction program module 220 of the computer server 210 creating the multivariate model which determines vulnerability to associated diseases at different stages of a disease being treated and an impact of surrounding context and current treatment with respect to the associated diseases, using the information received at steps 400, 410, and 420, the correlations from step 430, the clusters from step 300 (of FIG. 3), the individual risk factors determined at step 310 (of FIG. 3), and the common risk factors determined at step 320 (of FIG. 3). The disease vulnerability prediction program module 220 then stores the multivariate model which determines vulnerabilities and impacts in the knowledge corpus 230 of the computer server 210.

FIG. 5 depicts a flowchart of an exemplary method for using the multivariate model (created according to the method of FIG. 4) to predict the possibility of two diseases occurring together in a patient performed by the disease vulnerability prediction program module 220 of the computer server 210 in accordance with aspects of the invention. The steps of the method are performed in the environment of FIG. 2 and are described with reference to the elements shown in FIG. 2.

At step 500, the computer server 210 receives detailed information about a patient. In embodiments, step 500 comprises the disease vulnerability prediction program module 220 of the computer server 210 receiving detailed information (e.g., age, demographic information, current condition, and contextual parameters such as current surroundings, geographic location, interaction with other people, etc.) about a patient that is being treated for a disease from the disease vulnerability prediction user interface program module 250 of one of the client computing devices 240-1, 240-2, . . . , 240-n.

At step 510, the computer server 210 determines a vulnerability score of the patient's current disease to each of one or more associated diseases using the knowledge corpus 230. In embodiments, step 510 comprises the disease vulnerability prediction program module 220 of the computer server 210 using the detailed information received at step 500 and the knowledge corpus 230 (generated at steps 430 and 440 of FIG. 4) to determine a predicted vulnerability of the patient having the current disease to associated diseases. In particular, in embodiments, the disease vulnerability prediction program module 220 uses machine learning techniques to analyze the detailed information received at step 500 to extract contextual parameters including physiological indicators, psychological attributes and insights, and past history. The disease vulnerability prediction program module 220 then uses the extracted contextual parameters and the multivariate model created and stored by the disease vulnerability prediction program module 220 in the knowledge corpus 230 (of FIG. 2) according to the method of FIG. 4 to determine a vulnerability score for one or more associated diseases. In embodiments, for each of the one or more associated diseases, the vulnerability score is a probability of the associated disease occurring in the patient, given the contextual parameters.

At step 520, the computer server 210 determines surrounding context (e.g., geographic location, exercise, diet, etc.) to be adopted and surrounding context to be avoided using the knowledge corpus 230. In embodiments, step 520 comprises the disease vulnerability prediction program module 220 of the computer server 210 using the detailed information received at step 500, the extracted contextual parameters from step 510, and the knowledge corpus 230 (generated at steps 430 and 440 of FIG. 4) to determine a surrounding context to be adopted and a surrounding context to be avoided. In an example, the disease vulnerability prediction program module 220 identifies a particular geographic location, exercise, and/or diet that reduces the likelihood of one or more associated diseases occurring in the patient. The disease vulnerability prediction program module 220 generates and/or displays a recommendation to the patient, doctor, medical staff, or family members for adopting the particular geographic location, exercise, and/or diet that reduces the likelihood of one or more associated diseases occurring in the patient. In another example, the disease vulnerability prediction program module 220 identifies a particular geographic location, exercise, and/or diet that increases the likelihood of one or more associated diseases occurring in the patient. The disease vulnerability prediction program module 220 generates and/or displays a recommendation to the patient, doctor, medical staff, or family members for avoiding the particular geographic location, exercise, and/or diet that increases the likelihood of one or more associated diseases occurring in the patient.

At step 530, the computer server 210 determines treatments (e.g., a medicine and dosage) to be adopted and treatments to be avoided using the knowledge corpus 230. In embodiments, step 530 comprises the disease vulnerability prediction program module 220 of the computer server 210 using the detailed information received at step 500, the extracted contextual parameters from step 510, and the knowledge corpus 230 (generated at steps 430 and 440 of FIG. 4) to determine treatments to be adopted and treatments to be avoided. Based on the determined treatments to be adopted and the treatments to be avoided, as well as a patient's response to current treatment, the disease vulnerability prediction program module 220 adjusts a current treatment plan. In embodiments, the disease vulnerability prediction program module 220 generates and/or displays a recommendation to the doctor or medical staff for the adjusted treatment plan and/or the determined treatments to be adopted and treatments to be avoided.

Accordingly, it is understood from the foregoing description that embodiments of the invention provide a method for identifying common risk factors associated with two or more diseases; determining diseases that can occur together based on the identified common risk factors and through a correlation clustering data pipeline; and predicting a vulnerability score for a user's likelihood of two or more diseases occurring in the user based on contextual parameters that include physiological indicators of the user, psychologic attributes and insights of a user, and user history. Additionally, in embodiments, a current treatment plan is adjusted using machine learning techniques based on user response to the current treatment. Additionally, in embodiments, a display is created of the predicting possibility along with contextual information that should be avoided or adopted to avoid other diseases with the user.

In embodiments, a service provider could offer to perform the processes described herein. In this case, the service provider can create, maintain, deploy, support, etc., the computer infrastructure that performs the process steps of the invention for one or more customers. These customers may be, for example, any business that uses cloud computing technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system/server 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system/server 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
determining, by a computing device, information about a patient including a current disease and contextual parameters;
determining, by the computing device, a vulnerability score indicating a predicted vulnerability of the patient to at least one associated disease, based on the information about the patient;
determining, by the computing device, a surrounding context for the patient, based on the information about the patient; and
determining, by the computing device, a treatment for the patient, based on the information about the patient.

2. The computer-implemented method according to claim 1, wherein the contextual parameters include physiological indicators for the patient.

3. The computer-implemented method according to claim 1, wherein the contextual parameters include psychological attributes and insights for the patient.

4. The computer-implemented method according to claim 1, wherein the contextual parameters include past history for the patient.

5. The computer-implemented method according to claim 1, wherein the determining the surrounding context for the patient comprises determining a surrounding context to be adopted and a surrounding context to be avoided.

6. The computer-implemented method according to claim 5, wherein the surrounding context includes a geographic location.

7. The computer-implemented method according to claim 1, wherein the determining the treatment for the patient comprises determining a treatment to be adopted and a treatment to be avoided.

8. A computer program product comprising a computer readable storage medium having program instructions embodied therewith, the program instructions executable by a computing device to cause the computing device to:
determine diseases from a plurality of diseases that commonly occur together using clustering;
determine individual risk factors of each of the plurality of diseases using a data pipeline;
determine common risk factors of the plurality of diseases; and
determine a probability of two diseases occurring together in a patient using the determined diseases that commonly occur together, the determined individual risk factors, and the determined common risk factors.

9. The computer program product according to claim 8, wherein the determining the diseases from the plurality of diseases that commonly occur together comprises finding clusters between contextual parameters and the diseases.

10. The computer program product according to claim 9, wherein the contextual parameters include physiological indicators for the patient.

11. The computer program product according to claim 9, wherein the contextual parameters include psychological attributes and insights for the patient.

12. The computer program product according to claim 9, wherein the contextual parameters include past history for the patient.

13. The computer program product according to claim 9, wherein the determining the individual risk factors comprises using a logistic model formula to determine a probability of a selected disease as a function of a value of the contextual parameters.

14. The computer program product according to claim 13, wherein the logistic model formula is
$$y = log [ \frac{p ( X )}{1 - p ( X )} ] = {β}_{0} + {β}_{1} {x}_{1} + {β}_{2} {x}_{2} + , … , + {β}_{k} {x}_{k} .$$

15. A system comprising:
a hardware processor, a computer readable memory, and a computer readable storage medium associated with a computing device;
program instructions to determine information about a patient including a current disease and contextual parameters;
program instructions to determine a vulnerability score indicating a predicted vulnerability of the patient to at least one associated disease, based on the information about the patient;
program instructions to determine a surrounding context for the patient, based on the information about the patient; and
program instructions to determine a treatment for the patient, based on the information about the patient,
wherein the program instructions are stored on the computer readable storage medium for execution by the hardware processor via the computer readable memory.

16. The system according to claim 15, wherein the contextual parameters include physiological indicators for the patient.

17. The system according to claim 15, wherein the contextual parameters include psychological attributes and insights for the patient.

18. The system according to claim 15, wherein the contextual parameters include past history for the patient.

19. The system according to claim 15, wherein the determining the surrounding context for the patient comprises determining a surrounding context to be adopted and a surrounding context to be avoided.

20. The system according to claim 15, wherein the determining the treatment for the patient comprises determining a treatment to be adopted and a treatment to be avoided.
